(19) United States
(12) Patent Application Publication
(10) Pub. No.: US 2024/0038385 A1
(43) Pub. Date: Feb. 1, 2024

(54) AT-HOME DRUG TESTING

(71) Applicant: EMED LABS, LLC, Miami, FL (US)

(72) Inventor: Zachary Carl Nienstedt, Wilton Manors, FL (US)

(21) Appl. No.: 18/362,858

(22) Filed: Jul. 31, 2023

Related U.S. Application Data
(60) Provisional application No. 63/369,847, filed on Jul. 29, 2022.

(51) Int. Cl. G16H 40/67; G16H 80/00; G16H 15/00; G09B 19/00; G06V 20/40; G06V 20/52

(52) U.S. Cl. CPC G16H 40/67; G16H 80/00; G16H 15/00; G09B 19/003; G06V 20/40; G06V 20/52; G06Q 50/265

(57) ABSTRACT

Systems, methods, and graphical user interfaces for remotely proctored, at-home drug testing are disclosed herein. In some embodiments, a video connection, an audio connection, and/or a real-time messaging service may be established between a user device and a supervising user device for the purposes of conducting remote drug testing. User interface data may be sent to the user device to display various instructions to assist in the remote drug testing, such as instructions for providing an image of user identification or instructions for collecting a sample or developing results. User interface data may also be sent to the supervising user device for the purposes of supervising the remote drug testing, such as for verifying the user identification or recording the results.

### CROSS-REFERENCE TO RELATED APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/369,847, entitled “AT-HOME DRUG TESTING METHOD,” filed Jul. 29, 2022, the contents of which are incorporated by reference herein in their entirety.

### TECHNICAL FIELD

The embodiments of the disclosure generally relate to systems, methods, and graphical user interfaces for remotely proctored, at-home drug testing.

### BACKGROUND

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

A remotely proctored drug test system and method for at-home drug testing may overcome test security and integrity concerns while providing convenience and privacy to the test taker by providing a virtual proctored testing session wherein a test taker is observed during some or all of the testing by a remote proctor.

Systems, methods, and graphical user interfaces for remotely proctored, at-home drug testing are disclosed herein. In some embodiments, a video connection, an audio connection, and/or a real-time messaging service may be established between a user device and a supervising user device for the purposes of conducting remote drug testing. User interface data may be sent to the user device to display various instructions to assist in the remote drug testing, such as instructions for providing an image of user identification or instructions for collecting a sample or developing results. User interface data may also be sent to the supervising user device for the purposes of supervising the remote drug testing, such as for verifying the user identification or recording the results.

In some aspects, the techniques described herein relate to a computer-implemented method for remote drug testing, the method including: receiving, from a user device, first user input data verifying that a user has not performed oral consumption for a period of time; directing the user device to perform a check of components involved in the remote drug testing; sending, to the user device, first user interface data for displaying instructions for the user to follow prior to beginning the remote drug testing; receiving, from the user device, second user input data initiating the remote drug testing; upon receiving the second user input data, establishing a video connection, an audio connection, and a real-time messaging service between the user device and a supervising user device; sending, to the user device, second user interface data for displaying instructions to provide an image of a user identification via their user device; facilitating transmittal, from the user device to the supervising user device, the image of the user identification; receiving, from the supervising user device, a verification of the user identification; sending, to the user device, an image of a test; receiving, from the user device, third user input data confirming that the test matches a user test; facilitating transmittal, from the user device to the supervising user device, an image of the user test; receiving, from the supervising user device, a confirmation that the user test is not expired; sending, to the user device, third user interface data for displaying instructions to collect a sample and instructions to develop results of the user test from the sample; facilitating transmittal, from the user device to the supervising user device, results of the user test; receiving, from the supervising user device, a recordation of the results of the user test; generating a report based on the recordation of the results of the user test; and sending the report to the user device.

In some aspects, the techniques described herein relate to a computer-implemented method, wherein the supervising user device is a proctor device. In some aspects, the techniques described herein relate to a computer-implemented method, wherein the user identification is an identification card for the user. In some aspects, the techniques described herein relate to a computer-implemented method, wherein the method further includes: sending, to the user device, instructions illustrating components of the user test and explaining how the components will be used. In some aspects, the techniques described herein relate to a computer-implemented method, wherein the method further includes: sending the report to an employer, government agency, or an insurance agency.

In some aspects, the techniques described herein relate to a computer-implemented method, wherein the method further includes: collecting video data from the user device during the remote drug testing; and analyzing the video data to check that the user test is within view of a camera of the user device throughout the remote drug testing. In some aspects, the techniques described herein relate to a computer-implemented method, wherein the method further includes: collecting video data from the user device during the remote drug testing; analyzing the video data to check that the user test is within view of a camera of the user device throughout the remote drug testing; and upon determining that the user test is not within view of the camera of the user device throughout the remote drug testing, invalidating the results of the user test.

In some aspects, the techniques described herein relate to a computer-implemented method, wherein the method further includes: sending, to the supervising user device, fourth user interface data for verifying the user identification. In some aspects, the techniques described herein relate to a computer-implemented method, wherein the method further includes: sending, to the supervising user device, fourth user interface data for confirming that the user test is not expired. In some aspects, the techniques described herein relate to a computer-implemented method, wherein the method further includes: sending, to the supervising user device, fourth user interface data for recording the results of the user test.

In some aspects, the techniques described herein relate to a non-transient computer readable medium containing program instructions for causing a computer to perform a method for remote drug testing, the method including: receiving, from a user device, first user input data verifying that a user has not performed oral consumption for a period of time; directing the user device to perform a check of components involved in the remote drug testing; sending, to the user device, first user interface data for displaying instructions for the user to follow prior to beginning the remote drug testing; receiving, from the user device, second user input data initiating the remote drug testing; upon receiving the second user input data, establishing a video connection, an audio connection, and a real-time messaging service between the user device and a supervising user device; sending, to the user device, second user interface data for displaying instructions to provide an image of a user identification via their user device; facilitating transmittal, from the user device to the supervising user device, the image of the user identification; receiving, from the supervising user device, a verification of the user identification; sending, to the user device, an image of a test; receiving, from the user device, third user input data confirming that the test matches a user test; facilitating transmittal, from the user device to the supervising user device, an image of the user test; receiving, from the supervising user device, a confirmation that the user test is not expired; sending, to the user device, third user interface data for displaying instructions to collect a sample and instructions to develop results of the user test from the sample; facilitating transmittal, from the user device to the supervising user device, results of the user test; receiving, from the supervising user device, a recordation of the results of the user test; generating a report based on the recordation of the results of the user test; and sending the report to the user device.

In some aspects, the techniques described herein relate to a non-transient computer readable medium, wherein the supervising user device is a proctor device. In some aspects, the techniques described herein relate to a non-transient computer readable medium, wherein the user identification is an identification card for the user. In some aspects, the techniques described herein relate to a non-transient computer readable medium, wherein the method further includes: sending, to the user device, instructions illustrating components of the user test and explaining how the components will be used. In some aspects, the techniques described herein relate to a non-transient computer readable medium, wherein the method further includes: sending the report to an employer, government agency, or an insurance agency.

In some aspects, the techniques described herein relate to a non-transient computer readable medium, wherein the method further includes: collecting video data from the user device during the remote drug testing; and analyzing the video data to check that the user test is within view of a camera of the user device throughout the remote drug testing. In some aspects, the techniques described herein relate to a non-transient computer readable medium, wherein the method further includes: collecting video data from the user device during the remote drug testing; analyzing the video data to check that the user test is within view of a camera of the user device throughout the remote drug testing; and upon determining that the user test is not within view of the camera of the user device throughout the remote drug testing, invalidating the results of the user test.

In some aspects, the techniques described herein relate to a non-transient computer readable medium, wherein the method further includes: sending, to the supervising user device, fourth user interface data for verifying the user identification. In some aspects, the techniques described herein relate to a non-transient computer readable medium, wherein the method further includes: sending, to the supervising user device, fourth user interface data for confirming that the user test is not expired. In some aspects, the techniques described herein relate to a non-transient computer readable medium, wherein the method further includes: sending, to the supervising user device, fourth user interface data for recording the results of the user test.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 shows an example of a user's mobile display screen when the user launches the remotely proctored drug test system 1.

FIG. 2 shows an example of a user's mobile display screen when the user is prompted to log-in

FIG. 3 shows an example of a user's mobile display screen when the user is queried for additional information.

FIG. 4 shows an example of a user's mobile display screen where the user has responded to a query from the remote proctor system.

FIG. 5 shows an example of a user's mobile display screen when the remotely proctored drug test system verifies that all components of the system are working correctly.

FIG. 6 shows an example of a user's mobile display screen when the user is provided pre-test messages and instructions.

FIG. 7 shows an example of a user's mobile display screen as the user device is remotely connected with a remote proctor's device.

FIG. 8 shows an example of a user's mobile display screen when connection is established between a user and a remote proctor.

FIG. 9 shows an example of a user's mobile display screen when the user's identity is being verified by a remote proctor.

FIG. 10 shows an example of a user's mobile display screen when a drug test is being verified by a remote proctor.

FIG. 11 shows an example of a user's mobile display screen when a remote proctor is monitoring the user unpacking the drug test kit.

FIG. 12 shows an example of a user's mobile display screen as a remote proctor is instructing the user to confirm that all drug test kit components are present.

FIGS. 13A-13F show examples of a user's mobile display screen as the user is guided through the steps of a sample collection.

FIG. 14A-14B show examples of a user's mobile display screen as the user is guided through the steps of reading and recording their results.

FIG. 15 shows an example of a user's mobile display screen when the live testing session concludes.

FIG. 16 shows an example of a user's mobile display screen when a post-test survey is provided to a user.

FIGS. 17A-17D show examples of a user's mobile display screen as the user receives and views their e-mailed test results on their email.

FIGS. 18A-18B show example side-by-side views of the proctor device display and the user device display during the start of the live testing session.

FIGS. 19A-19B show example side-by-side views of the proctor device display and the user device display once a connection is made between the proctor device and the user device.

FIGS. 20A-20B show example side-by-side views of the proctor device display and the user device display during the start of the testing experience.

FIGS. 21A-1B show example side-by-side views of the proctor device display and the user device display during the start of the testing experience.

FIGS. 22A-22B show example side-by-side views of the proctor device display and the user device display as the user is being queried for additional input.

FIGS. 23A-23B show example side-by-side views of the proctor device display and the user device display during the user identity verification portion of the testing experience.

FIGS. 24A-24B show example side-by-side views of the proctor device display and the user device display during the user identity verification portion of the testing experience.

FIGS. 25A-25B show example side-by-side views of the proctor device display and the user device display as the user is instructed how to set up for the testing experience.

FIGS. 26A-26B show example side-by-side views of the proctor device display and the user device display as a drug test kit expiration date is being verified.

FIGS. 27A-27B shows example side-by-side views of the proctor device display and the user device display as the components of a drug test kit are being verified.

FIGS. 28A-28F show example side-by-side views of the proctor device display and the user device display during the sample collection stage of the testing experience.

FIGS. 29A-29F show example side-by-side views of the proctor device display and the user device display during the sample testing stage of the testing experience.

FIGS. 30A-30F show example side-by-side views of the proctor device display and the user device display during the results interpretation stage of the testing experience.

FIGS. 31A-31B show example side-by-side views of the proctor device display and the user device display at the conclusion of the testing experience.

FIG. 32 is a block diagram illustrating an embodiment of a computer system configured to perform the identification verification methods described herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail below with reference to the example embodiments provided in the figures, this application describes systems, methods, and graphical user interfaces for remotely proctored, at-home drug testing. Embodiments of the inventions described herein can comprise several novel features, and no single feature is solely responsible for the desirable attributes or is essential to practicing the inventions described.

There are many instances when an individual may be required to take a mandatory drug test, such as part of a pre-employment screening, after being selected for a random drug screening at their place of employment, before participating in an athletic event, or to show compliance with terms of parole or probation. Important decisions that involve consequential aspects of the test taker's future may be made based on the results of the test. Thus, the organizations that require drug testing must be certain that the drug test is performed securely and without tampering so that the results of the test can be trusted as a basis for making important decisions.

Current options for drug testing are limited and inconvenient for test takers. Often, drug tests are only administered in a lab on certain days or at certain times (e.g., Wednesday afternoons between 1 PM and 4 PM). This requires the test taker must show up at the lab at the specified day and time, which may force them to rearrange their schedules or take time off of work. The user must then often wait in a waiting room along with every other test taker for that day. Lines can be long and wait times may result in hours of wasted time for the test taker. Unfortunately, test takers currently do not have much choice but to endure such inconveniences when required to take a mandatory drug test. Despite its availability and convenience, at-home drug testing has not been widely used because of its inability to guarantee test security and results validity.

A remotely proctored drug test system and method for home drug testing is proposed that overcomes the test security problems described while providing convenience and privacy to the test taker. The system may include a proctored testing session wherein a test taker (e.g., a user) is observed during some or all of the testing by a remote proctor (e.g., a supervising user). In some embodiments, the remotely proctored drug test system may be implemented in a web browser or a web-based application on a computing or mobile device (e.g., smartphone, tablet, etc.). For the purposes of facilitating understanding, in some embodiments, the device of a user (e.g., a test taker) may be referred to as a user device and the device of a supervising user (e.g., a proctor) may be referred to as a supervising user device.

The test flow may include various steps, which will be described herein. However, it should be understood that any particular step may be optional and/or the steps may be performed in different orders.

In some embodiments, a test taker (e.g., a user) launches a web- or application-based testing experience on a user device. In some embodiments, a test taker creates or logs in to an account.

In some embodiments, a system requests input from the test taker prior to being connected to a proctor (e.g., a supervising user). In some embodiments, the system provides graphical user interface data to the user device for collecting this user input. For example, test taker may verify that they have not consumed food, drink, tobacco or other products (e.g., medication, gum, toothpaste, etc.) for a certain period of time (e.g., 10 minutes) prior to the testing to minimize chances of the test results being invalidated. In some embodiments, the system may request other information such as why the user is taking the test (e.g., for work, for insurance, or for other reasons, etc.), demographic information, location information, whether this is the user's first time testing, etc.

In some embodiments, the system completes a check of components on the user device. Depending on the testing experience, different components may be checked. For example, the testing experience may include audio input/output, augmented reality, front- and/or rear-facing camera image collection, etc. Thus, the system may check to make sure it has access to components such as microphone, speakers, front- and rear-facing cameras, etc. The system may also check to make sure each of the components has sufficient input/output sound or image quality.

In some embodiments, the system provides any pre-test messaging/instructions to the user. In some embodiments, the system provides graphical user interface data to the user device for displaying the instructions to the user. This can include instructions for the user prior to beginning the test, an overview of what the test experience includes, a list of any additional items that the user will need to complete the testing experience, information about how test results will be handled, etc. The messaging/instructions may be delivered using one or more of text, audio, augmented reality/image/video-based graphics, etc. In some embodiments, once user has received the messaging/instructions, they may click a “connect” button to begin the test experience.

In some embodiments, the system connects the user device to a proctor device (e.g., a supervising user device), thereby establishing a connection between the test taker and the proctor (e.g., they are connected by audio, video, and/or real-time messaging). This may include establishing a video and/or audio connection between the user device and the proctor device. This may include establishing real-time messaging between the user device and the proctor device. In some embodiments, one or more of the user device camera or proctor device cameras may be turned off, such that there is one-way video feed. For example, the proctor device may receive video data from the test taker, but the test taker may not receive video data from the proctor device. Audio may be treated similarly (e.g., user device microphone can be muted while the proctor device microphone is on so that the test taker can hear the proctor speaking but the proctor cannot hear the test taker) or audio feed may be two-way to facilitate conversation between the test taker and the proctor.

In some embodiments, the test taker may be instructed to show identification (e.g., an image of a user identification card) to the proctor. In some embodiments, the system provides graphical user interface data to the user device for displaying the instructions to the user. The proctor device may receive the image of the identification from the user device. In some embodiments, the system may facilitate the transmission of the image of the identification from the user device to the proctor device. Computer vision tools may be used to identify/confirm important information on the ID (e.g., full name, expiration date, address, etc.). The computer vision tools may automatically import this information into a user profile or may ask the proctor to confirm accuracy.

In some embodiments, the system may perform test verification my showing the test taker an image of the test they will be taking. For instance, the system may send an image of the test to the user device. The test taker can compare the image to their own test (e.g., the user test) and confirm that they are the same. Confirmation may also be completed by asking the user to place their user test within view of the front- or rear-facing camera and an image can be captured and sent to the proctor. In some embodiments, the system may facilitate the transmission of the image of the user test from the user device to the proctor device. The proctor and/or a computer vision tool may check to make sure that the test taker's test is supported by the testing experience. An expiration date of the test may also be identified and checked to make sure the test is not expired. If the test is not expired, the user continues with the testing experience. In some embodiments, if the test is expired or not supported by the testing experience, the test taker may be directed to a shopping page where unexpired, supported tests may be purchased. This may conclude the proctor connection and the testing experience.

In some embodiments, the user test may be a test kit and the user may have to unpack the components of the test kit. In some embodiments, the system may illustrate the items that should be included in the test kit. The test taker, following proctor prompting, may open the test and arrange the test kit components such that they are within view of the front- or rear-facing camera on the user device. In some embodiments, the test taker may also be within view of the front- or rear-facing camera on the user device such that the proctor can see the test taker and the test during all or parts of the testing experience. In some embodiments, the system may be able to confirm that all test components are present. For example, the system may be able to illustrate which components are present in the test kit and explain to the user what they are and/or how they will be used. More specifically, the system may be able to send, to the user device, graphical user interface data for displaying instructions that illustrate components of the user test and explain how the components will be used.

In some embodiments, the system may be able to instruct the test taker through a sample collection or the development of test results from the sample. In some embodiments, images, videos, and/or augmented reality sample collecting instructions are presented to the test taker. For instance, the system may be able to send, to the user device, graphical user interface data for displaying images, videos, and/or augmented reality sample collecting instructions that instruct the test taker through a sample collection. The proctor may also provide audio instructions to the test taker. In some embodiments, once the sample is collected, instructions about placing the sample into the diagnostic portion of the test kit may be provided. The system may send graphical user interface data for displaying the instructions to the user device. For many tests, the test taker must wait a certain amount of time while the sample is being tested and results are developing. The system may start a timer once the user has placed their sample into the diagnostic portion of the test. The proctor may disconnect from the test taker during this timed portion of the test. Video and/or audio from the user may still be collected so that the test is visible to the system at all times. This discourages test tampering and, if any tampering does occur, it will be captured by the system for later viewing. In some embodiments, test results may be invalidated if the test is not within view of the user device camera throughout the entire test experience.

In some embodiments, the system may be able to facilitate the reading and recording of test results by the proctor. Once the required amount of time has passed, results may be read and interpreted by the proctor (e.g., over the video connection). If the proctor previously disconnected, the same or a different proctor may reconnect to guide the test taker through this portion of the test. The proctor may show the test taker pictures of positive, negative, and/or invalid results to help the test taker read their results. In some embodiments, computer vision tools may be used to automatically read the test results. The proctor and/or test taker may then confirm that the CV reading is correct or may disagree with the CV reading if needed. In some embodiments, once the results are read, they may be recorded and populated into a report. In some embodiments, the system may send graphical user interface data to the proctor device for displaying a user interface that can be used by the proctor to record the various results (e.g., “negative”, “positive”, “invalid”, and so forth).

In some embodiments, the interpreted results and/or report can be displayed on the user device. In some embodiments, this may conclude the live testing experience and the proctor may disconnect. In some embodiments, a post-test survey may be provided to the test taker to collect feedback and/or to provide follow-up contact information to the test taker. In some embodiments, a results report may be sent to a test taker (e.g., via email or to the user device). In some embodiments, the report may also be sent to an employer, government agency, insurance agency, etc.

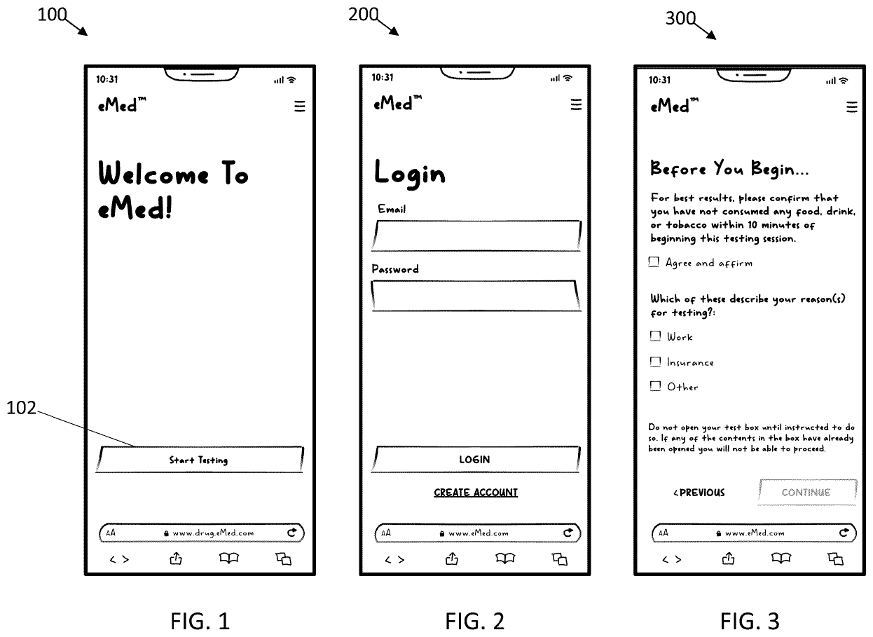

Turning now to the figures, FIG. 1 shows an example of a user's mobile display screen 100 when the user launches the remote proctor system. Upon launching the remote proctor system, there may be a prompt welcoming the user to the host of the remotely proctored drug test remote proctor system's (e.g., eMed) platform. Once the user is ready to begin the testing, they may press a start button 102, to take them to a log-in page where they are prompted to enter an email and password, such as the example screen 200 shown in FIG. 2. If the user does not have an account with the host of the remote proctor system, they may be prompted to create an account at this stage. Once the user is logged in, the remote proctor system may query the user for additional input prior to connecting the user to a remote proctor.

FIG. 3 shows an example of a user's mobile display screen 300 once the user is logged in and queried for additional information. As shown, the remote proctor system may ask the user to verify that they have not consumed food, drink, tobacco or other products (e.g., medication, gum, toothpaste, etc.) for a certain period of time (e.g., 10 minutes) prior to the testing to minimize chances of the test results being invalidated. In some embodiments, the remote proctor system may request other information, including but not limited to why the user is taking the test (e.g., for work, for insurance, or personal reasons, etc.), demographic information, location information, whether it is the user's first time testing, and the like. Querying the user for information such as and similar to the foregoing helps ensure the accuracy and integrity of the remote testing process.

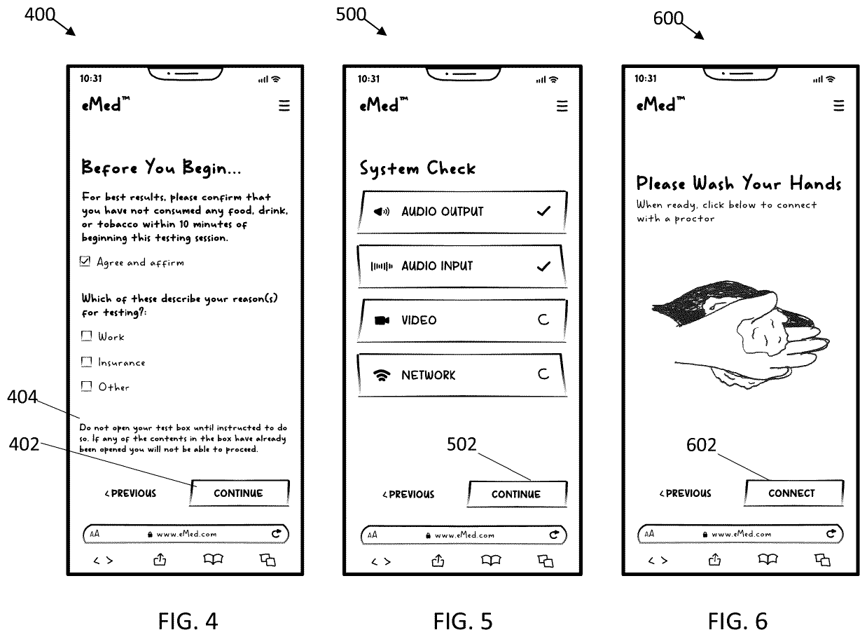

FIG. 4 shows an example embodiment of a user's mobile display screen 400 where the user has responded to a query from the remote proctor system. In some embodiments, the remote proctor system may present a notice 404 to the user, reminding them not to open their at-home test kit until instructed to by a virtual proctor as an additional measure to ensure test integrity. After the user has been queried for additional input, and been presented with the notice 404, they may indicate that they are ready to continue to the testing experience by pressing a Continue button 402.

FIG. 5 shows an example of a user's mobile display screen 500 as the remote proctor system verifies that all components working correctly. This process is performed by the remote proctor system as an additional measure to ensure test integrity. Depending on the testing experience, different components may be checked. For example, the testing experience may include audio input and output, augmented reality (“AR”), front- and/or rear-facing camera image collection, etc. Thus, the remote proctor system may check to make sure it has access to components such as microphone, speakers, front- and rear-facing cameras. The remote proctor system may also check to make sure each of the components has sufficient input and output sound and image quality. Once the remote proctor system has confirmed that all components work correctly, the user may proceed to the pre-test messages and instructions by pressing Continue button 502.

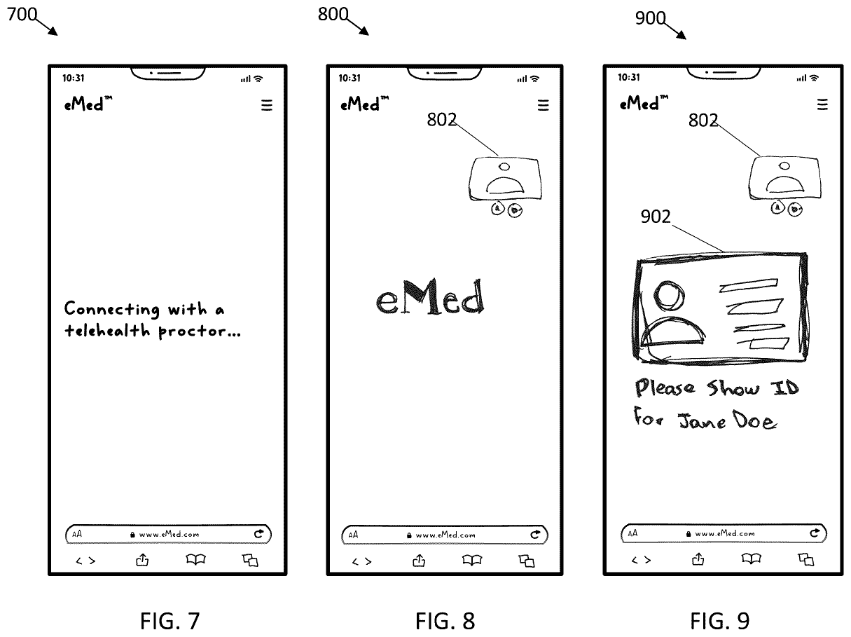

FIG. 6 shows an example embodiment of a user's mobile display screen 600 when the user is provided pre-test messages and instructions. In some embodiments, the pre-test messages and instructions may include an overview of what the testing experience includes, a list of any additional items that the user will need to complete the testing experience, information about how test results will be handled, and the like. The messages and instructions may be delivered using one or more of text, audio, AR, image or video-based graphics, and the like. Once user has received the pre-test messages and instructions, they may click a connect button 602 to begin the testing experience. The remote proctor system may then begin to establish a video and/or audio connection between the user device and the device of a remote proctor. FIG. 7 shows an example of a user's mobile display screen 700 as the user's device is remotely connected with a remote proctor's device.

FIG. 8 shows an example of a user's mobile display screen 800 when a remote connection is established between a user and a remote proctor 802. A user and a remote proctor 802 may be connected by one or more of audio, video, and real-time messaging. In some embodiments, one or more of the user device camera or proctor device camera may be turned off such there is one-way video feed. For example, the proctor device may receive video data from the user, but the user device may not receive video data from the remote proctor 802. Audio may be treated similarly—the user device microphone may be muted while the proctor device microphone is on such that the user may hear the remote proctor 802 speaking but the remote proctor 802 cannot hear the test taker. This feature may provide added privacy for the test taker. In some embodiments, audio feed may be two-way to facilitate conversation between the use and the remote proctor 802.

The testing experience may begin by verifying the user's identity. FIG. 9 shows an example of a user's mobile display screen 900 when the user's identity is being verified by a remote proctor. The user may be instructed to show their identification by the remote proctor 802 verbally, by real-time message, or both. In some embodiments, the remote proctor system may display the instructions to show identification to the proctor on the display. The user may show their identification to the remote proctor 802 either by sending a picture of their identification document 902 over real-time message or showing their identification document 902 to the remote proctor 802 over a live video feed. Once the proctor device receives an image or video of the user's their identification document 902, computer vision tools may be used to identify and confirm important information on the identification (e.g., full name, expiration date, address, etc.). The computer vision tools may automatically import this information into a user profile or may ask the remote proctor 802 to confirm accuracy of the information captured for added verification. Once the user's identity has been verified, the remote proctor 802 may proceed to verify the drug test kit.

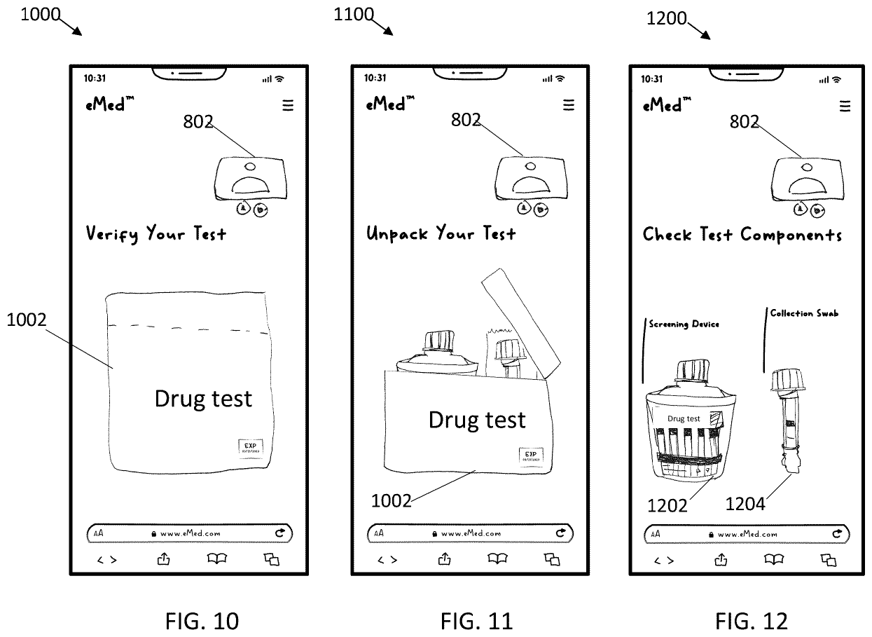

FIG. 10 shows an example of a user's mobile display screen 1000 when a drug test kit 1002 is being verified by the remote proctor 802. The remote proctor system may display to the user an image of the drug test kit corresponding to the drug test kit 1002 the user will be taking. The user may compare the image of the drug test kit to their own physical drug test kit 1002 in order to confirm that they are the same. In some embodiments, confirmation may also be completed by asking the user to place their drug test kit 1002 within view of the front- or rear-facing camera of the user device, and a still image or video image may be captured and sent to the proctor device. The remote proctor 802 or a computer vision tool may then check to confirm that the test taker's drug test kit 1002 is supported by the testing experience. An expiration date of the drug test may also be identified and checked at this stage in order to confirm that the test is not expired, in order to ensure the accuracy the test results. If the drug test kit is not expired, the user may continue with the testing experience. If the drug test kit is expired or not supported by the testing experience, the use may be directed to a shopping web page where unexpired, supported drug test kits may be purchased. This may conclude the remote proctor's 802 connection and the testing experience.

FIG. 11 shows an example of a user's mobile display screen 1100 when a remote proctor 802 is monitoring the user unpacking the drug test kit 1002. The remote proctor system illustrates the items that should be included in the test kit 1002. Following prompts from the remote proctor 802, the user may open the test and arrange the test kit components such that they are within view of the front- or rear-facing camera on the user device. In some embodiments, the user may also be within view of the front- or rear-facing camera on the user device such that the remote proctor 802 can see the test taker and the test during all or parts of the testing experience.

FIG. 12 shows an example of a user's mobile display screen 1200 as a remote proctor is instructing the user to confirm that all test components for the given drug test kit 1002 are present. The remote proctor 802 may concurrently verify that all test components of the given drug test kit are present, as they are visible on the proctor device via the front- or rear-facing camera of the user device. The remote proctor system may illustrate which components (e.g. screening device 1202, collection swab 1204, etc.) are present in the test kit and may explain to the user what each component is and how they will be used. The remote proctor system, the remote proctor 802, or both may then guide the user through the sample collection process. In some embodiments sample collecting instructions may be presented to the user by one or more of images, videos, audio, and AR.

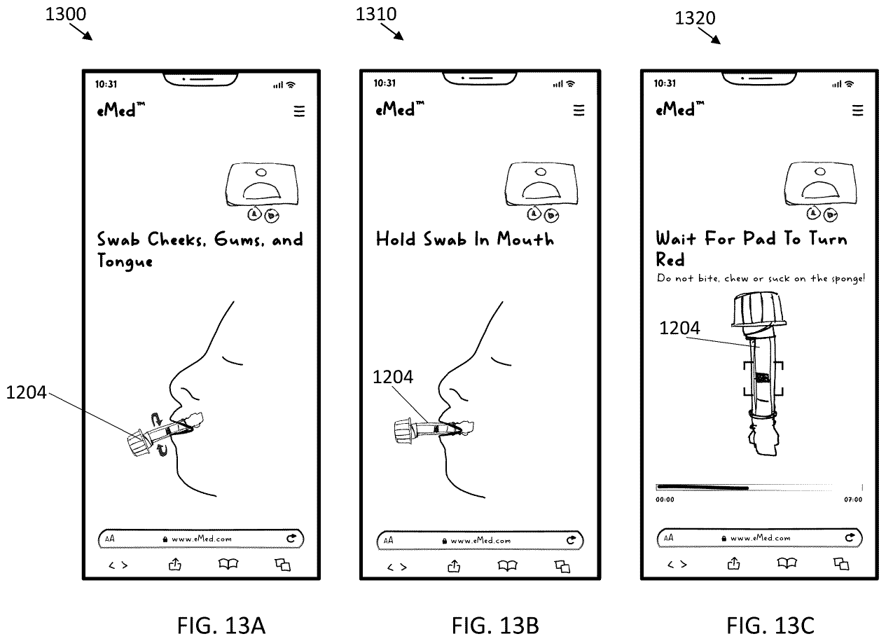

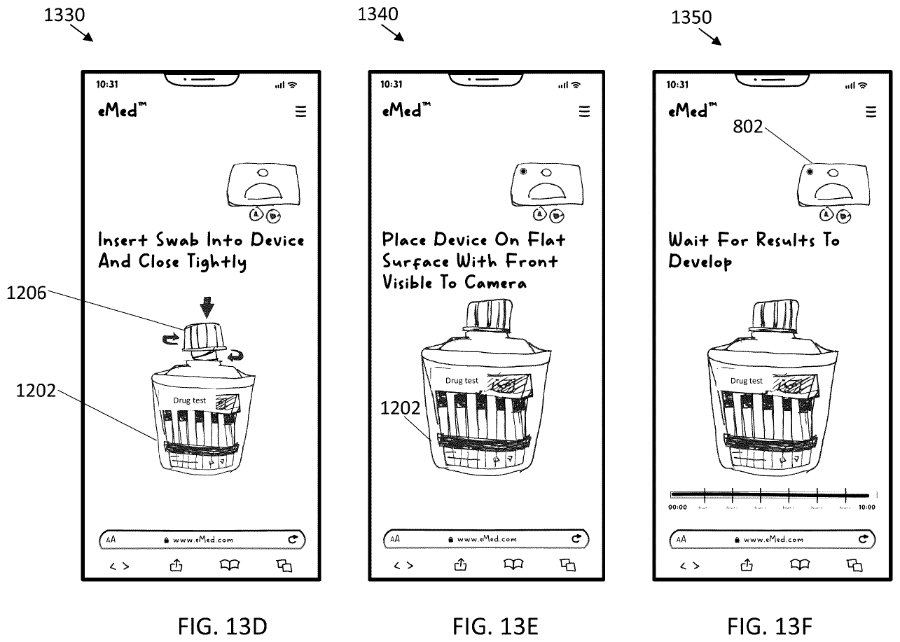

FIGS. 13A-F show examples of a user's mobile display screen as the user is guided through the steps of a sample collection. FIG. 13A shows an example of a user's mobile display screen 1300 with the user being instructed to swab their cheeks, gums and tongue with the collection swab 1204. FIG. 13B shows an example of a user's mobile display screen 1310 the user being instructed to hold the swab collection swab 1204 in their mouth. FIG. 13C shows an example of a user's mobile display screen 1320 with the user being instructed to wait for an indication pad on the collection swab 1204 to turn red, and to not bite, suck, or chew on the sponge portion of the collection swab 1204. Once the sample is collected, instructions about placing the sample into the diagnostic portion of the test kit may be provided. FIG. 13D shows an example of a user's mobile display screen 1330 with the user being instructed to insert the collection swab 1204 into a screening device 1202 of the drug test kit and to close the lid 1206 tightly. FIG. 13E shows an example of a user's mobile display screen 1340 with the user being instructed to place the screening device 1202 on a flat surface, visible to the front camera. For many tests, the user must wait a certain amount of time while the sample is being tested and results are developing. FIG. 13F shows an example of a user's mobile display screen 1350 with the user being instructed to wait for the results to develop. The remote proctor system may start a timer once the user has placed their sample into the diagnostic portion of the test. The remote proctor 802 may disconnect from the test taker during this timed portion of the test, but video, audio, or both from the user may still be collected so that the test is visible to the remote proctor system at all times during the waiting period to discourage test tampering. If any tampering does occur, video evidence would be captured by the remote proctor system for later viewing. In some embodiments, test results may be invalidated if the test is not within view of the user device camera throughout the entire testing experience. Once the waiting period is concluded, the remote proctor system may read and interpret the results.

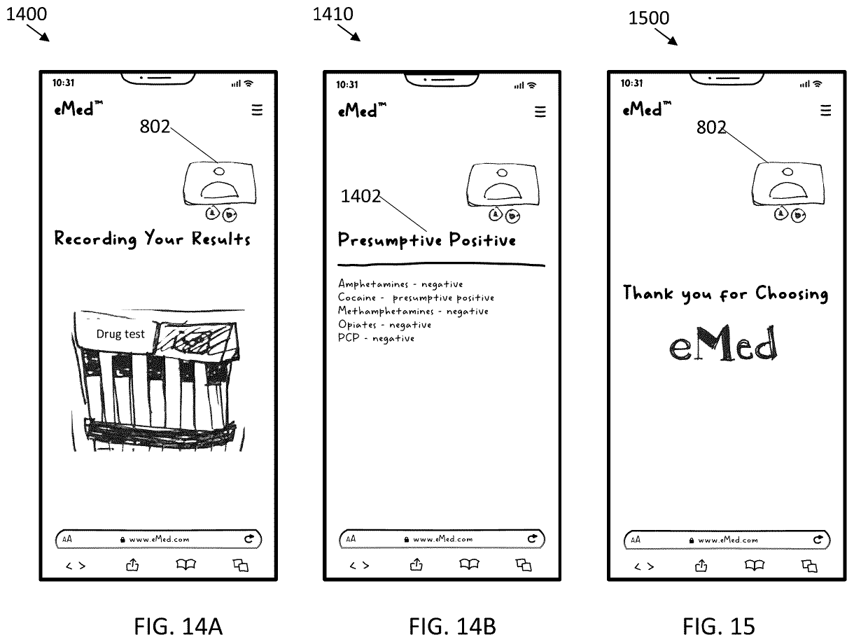

FIG. 14A-B show examples of a user's mobile display screen as the user is guided through the steps of reading and recording their results. FIG. 14A shows an example of a user's mobile display screen 1400 with the user being guided through the steps of reading and recording their results. If the remote proctor 802 previously disconnected, the same or a different proctor may reconnect to guide the user through this portion of the test. The remote proctor 802 may show the user pictures of positive, negative, and/or invalid results to help the test taker interpret their results as shown in FIG. 14A. In some embodiments, a computer vision tool may be used to automatically interpret the test results. The remote proctor 802, user, or both may then verify that the reading by the computer vision tool is correct or may disagree with the reading of the computer vision tool if it is incorrect. This may help improve the computer vision tool's artificial intelligence (“AI”) algorithm and improve the computer vision tool's accuracy. FIG. 14B shows an example of a user's mobile display screen 1410 with record results shown to the user. Once the results are read, they may be recorded and populated into a report 1402, such as one shown in FIG. 14B. The testing experience may then be concluded, and the user device and proctor device may be disconnected from each other. FIG. 15 shows an example of a user's mobile display screen 1500 when the live testing session concludes.

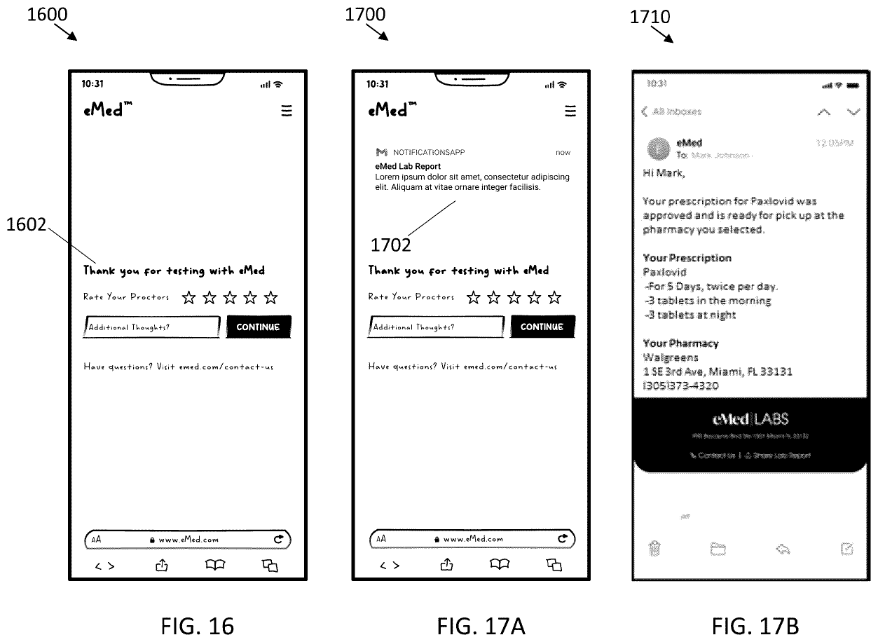

FIG. 16 shows an example of a user's mobile display screen 1600 when a post-test survey 1602 is provided to a user. In some embodiments, a post-test survey 1602 may be provided to the test taker to collect feedback and/or to provide follow-up contact information to the test taker.

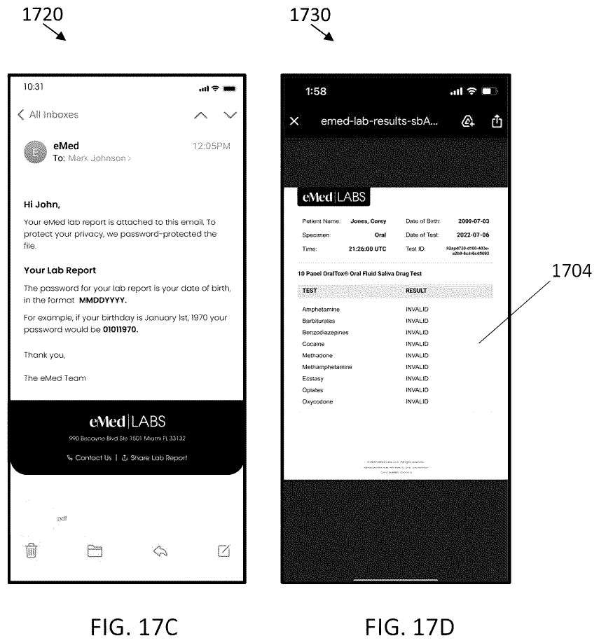

In some embodiments, the user's results report may be sent to the user by e-mail. FIG. 17A-17D show examples of a user's mobile display screen as the user receives and views their e-mailed test results on their email. In some embodiments, the results report may additionally be sent to an employer, government agency, insurance agency, and the like. FIG. 17A shows an example of a user's mobile display screen 1700 when the user receives a push notification 1702 that they have received an email with their test results. This email may be received within seconds of the conclusion of the testing experience. FIG. 17B shows an example of a user's mobile display screen 1710 with the user navigating to an email-application on their mobile device. In some cases, the user may be able to receive and read emails following the conclusion of the testing experience, such as reading a communication that their prescription was approved (e.g., because of their test results). FIG. 17C shows an example of a user's mobile display screen 1720 with an example message that would accompany the results. In some embodiments, the results may be attached to the email in a password-encrypted attachment for added security. FIG. 17D shows an example of a user's mobile display screen 1730 with an example of the results report 1704.

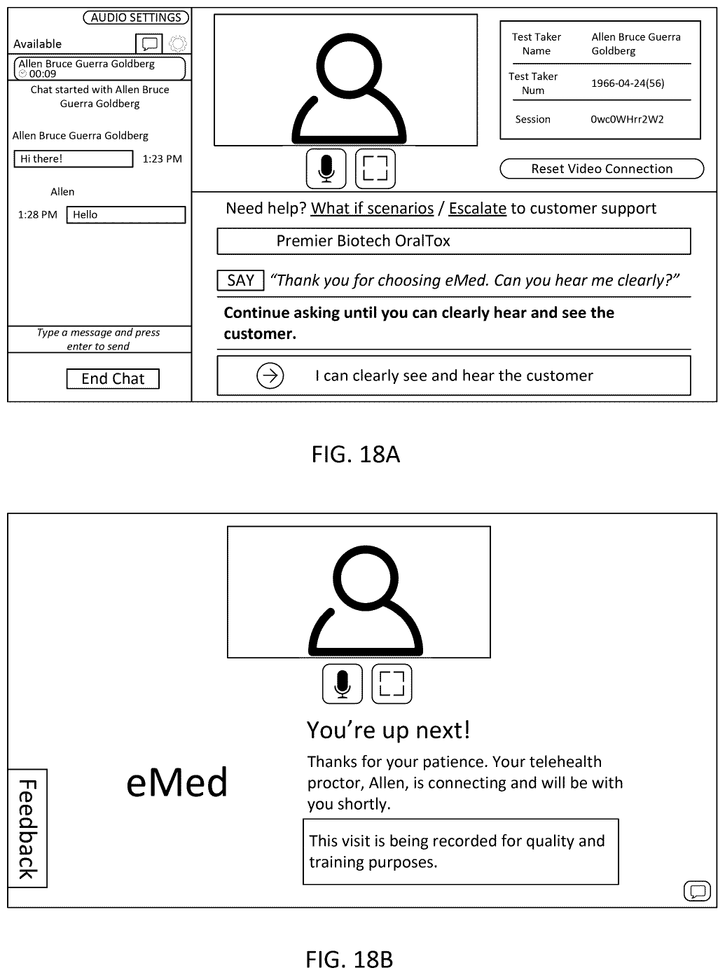

FIGS. 18A-18B show example side-by-side views of the proctor device display and the user device display once a connection is made between the proctor device and the user device. In some embodiments, there may be prompt for the proctor to confirm that the proctor and user are able see and hear each other clearly once a remote connection is established.

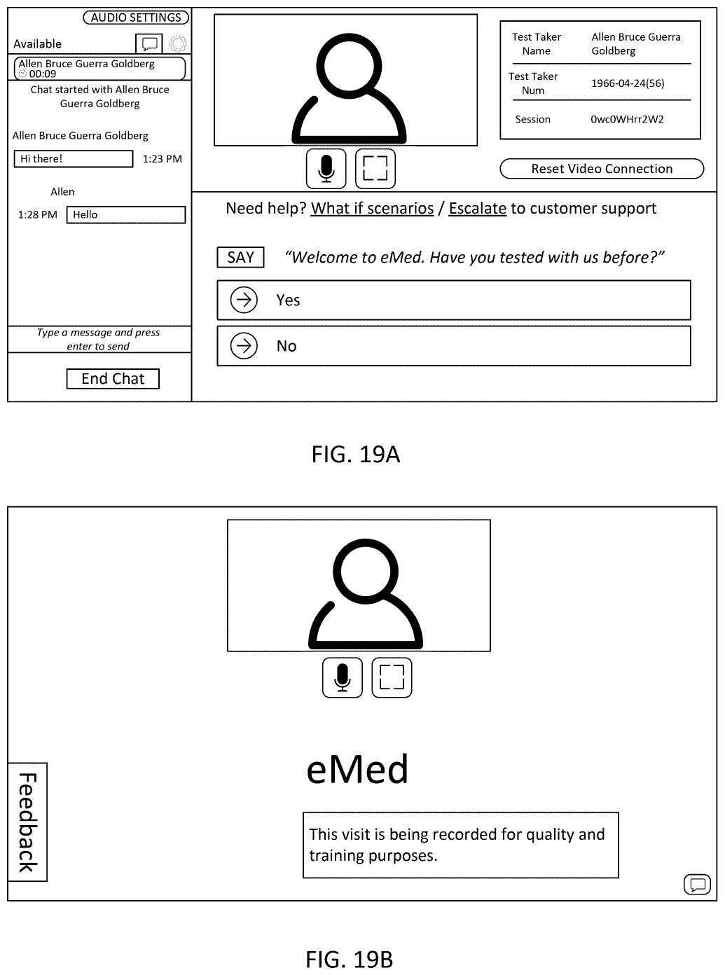

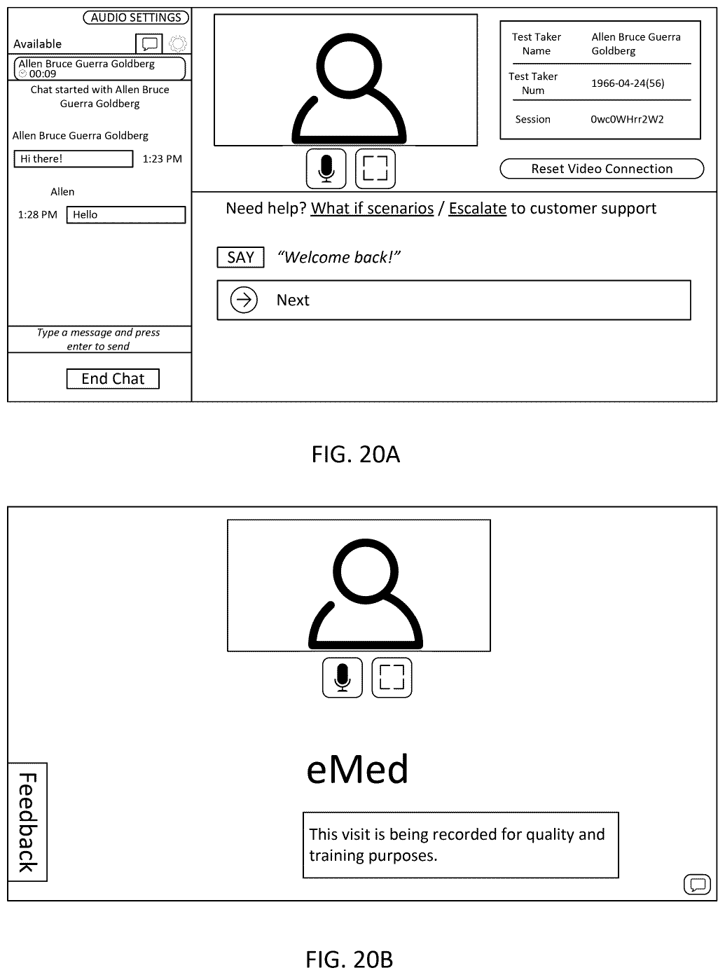

FIGS. 19A-19B show example side-by-side views of the proctor device display and the user device display during the start of the live testing session. In some embodiments, the proctor may start by asking the user if they have used the remote proctor system before, to gauge the user's familiarity with virtual testing. The proctor may enter the user's answer into the system in order to change the types of vocal prompts the instructor receives. For example, if the user answers “No” and the proctor enters that into the system, the next voice prompt for the proctor may be a detailed description of the overall testing process. If the user answers “Yes”, the remote proctor system may skip over the detailed description of the overall testing process since the user is already familiar with it. In this case, the proctor's prompt may instead be a simple “Welcome back!” as shown in FIGS. 20A-20B, which show example side-by-side views of the proctor device display and the user device display during the start of the testing experience.

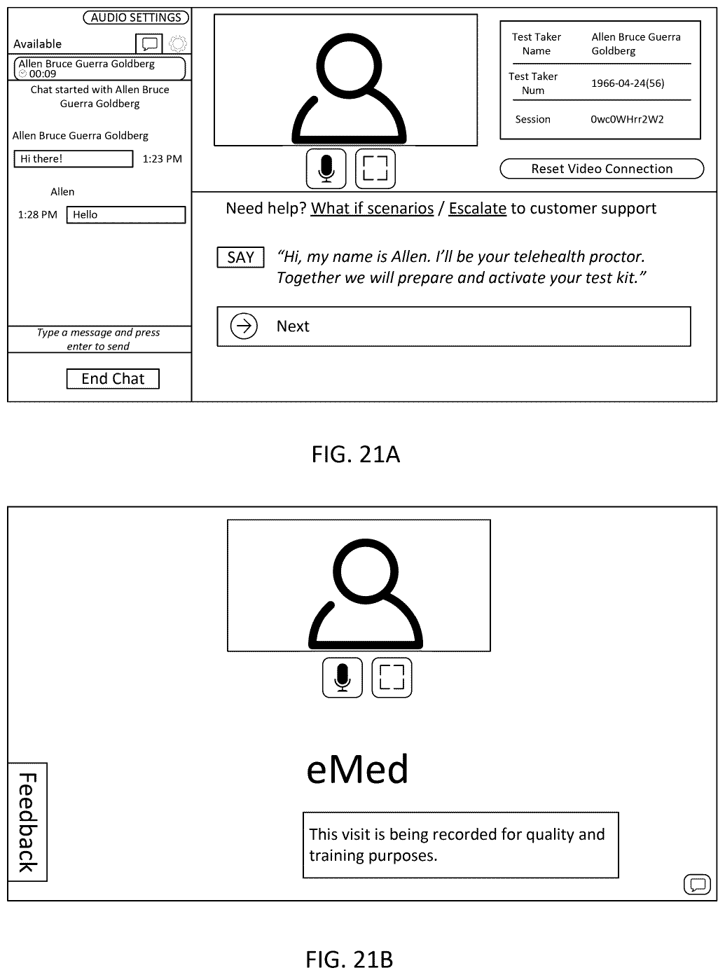

The proctor may then introduce themself and begin preparing for the testing process as shown in FIGS. 21A-21B, which show example side-by-side views of the proctor device display and the user device display during the start of the testing experience.

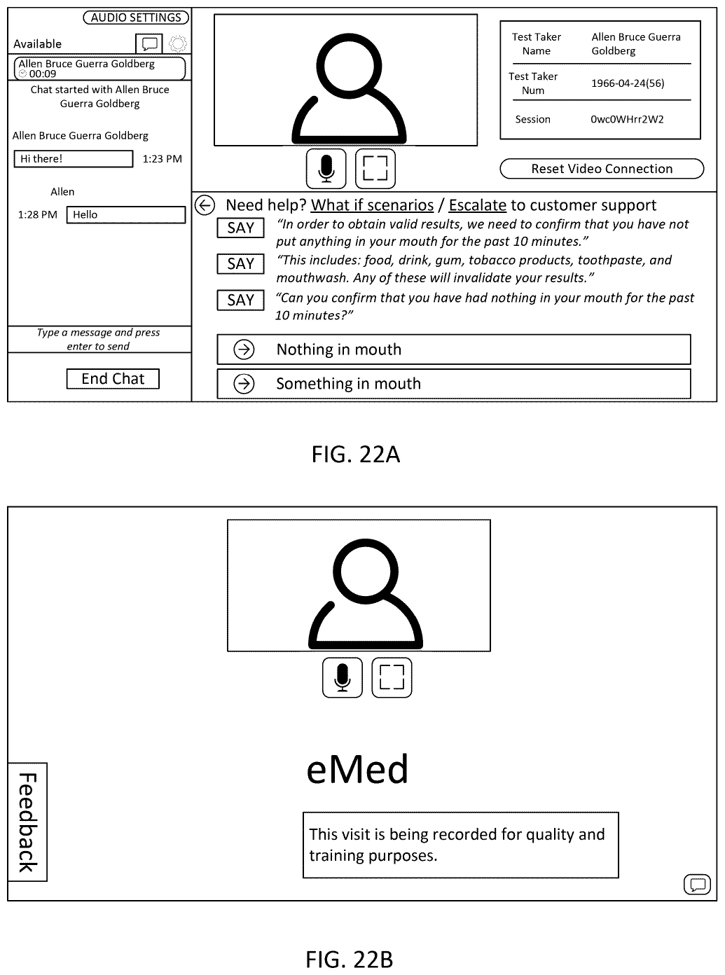

FIGS. 22A-2B show example side-by-side views of the proctor device display and the user device display as the user is being queried for additional input. In some embodiments, along with scripted prompts, the proctor may also be presented with interactive buttons so they may input the user's responses into the remote proctor system. The remote proctor system may use the user's answers into account when analyzing the test results.

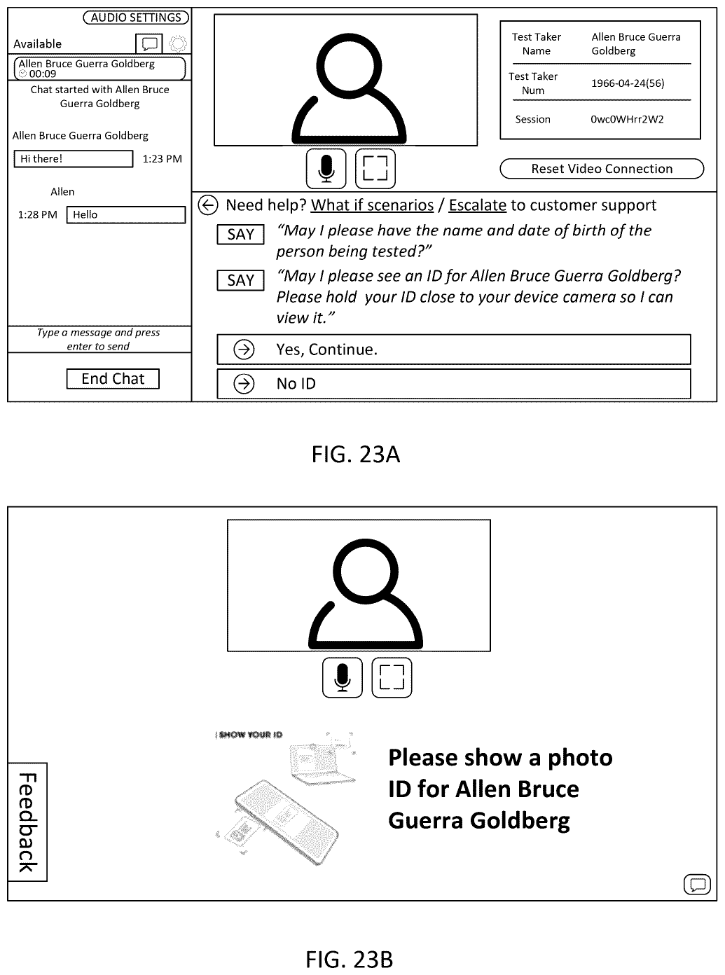

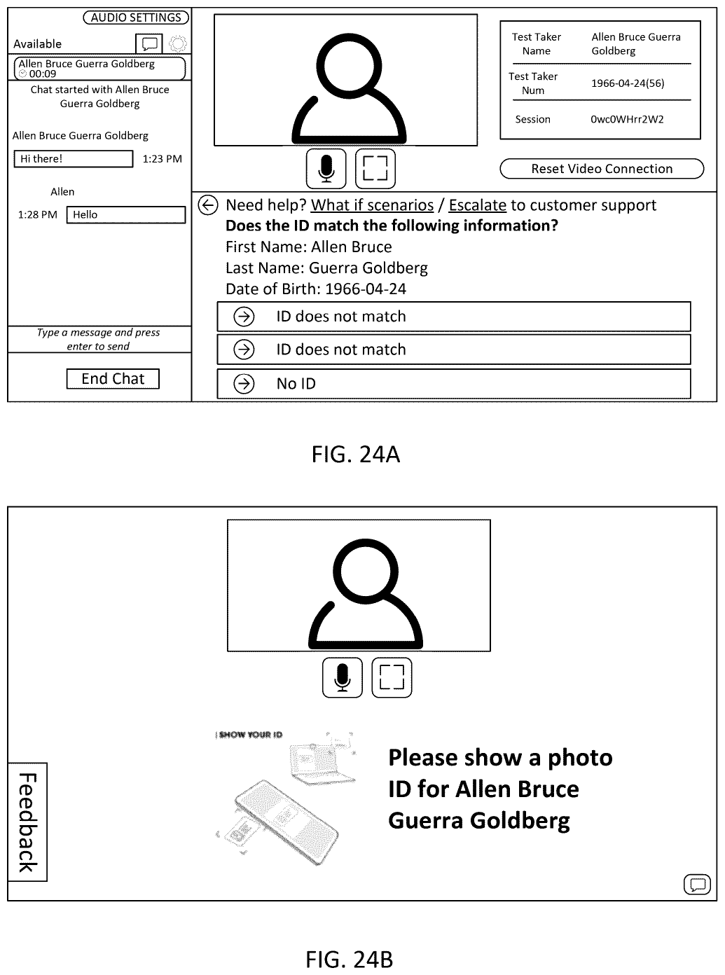

FIGS. 23A-3B show example side-by-side views of the proctor device display and the user device display during the user identity verification portion of the testing experience. The proctor may be prompted to ask the user for their name and date of birth, and to show their identification to a front- or rear-facing camera of the user device. In some embodiments, the user may also receive a message on their display instructing them to show their identification to a front- or rear-facing camera of the user device. Once the proctor receives an image of the user's identification, they may be prompted by the remote proctor system to confirm that the information on the identification correlated to the information in the remote proctor system, as shown in FIGS. 24A-24B, which show example side-by-side views of the proctor device display and the user device display during the user identity verification portion of the testing experience.

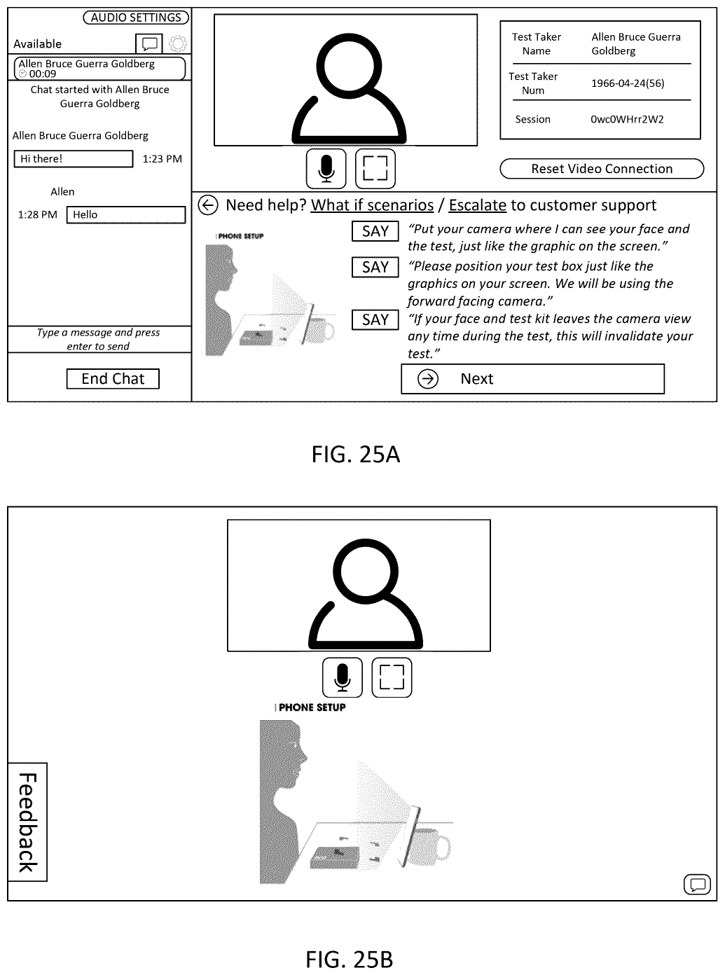

FIGS. 25A-25B show example side-by-side views of the proctor device display and the user device display as the user is instructed how to set up for the testing experience. In some embodiments, a graphic may be displayed on the user's display illustrating how to set up their device for testing. In some embodiments, the proctor may dictate instructions to the user such as “Put your camera where I can see your face and the test, just like the graphic on the screen”, “Please position your test box like the graphic on your screen”, “We will be using the forward-facing camera,” and “If your face and test kit leaves the camera view any time during the test, this will invalidate your test.”

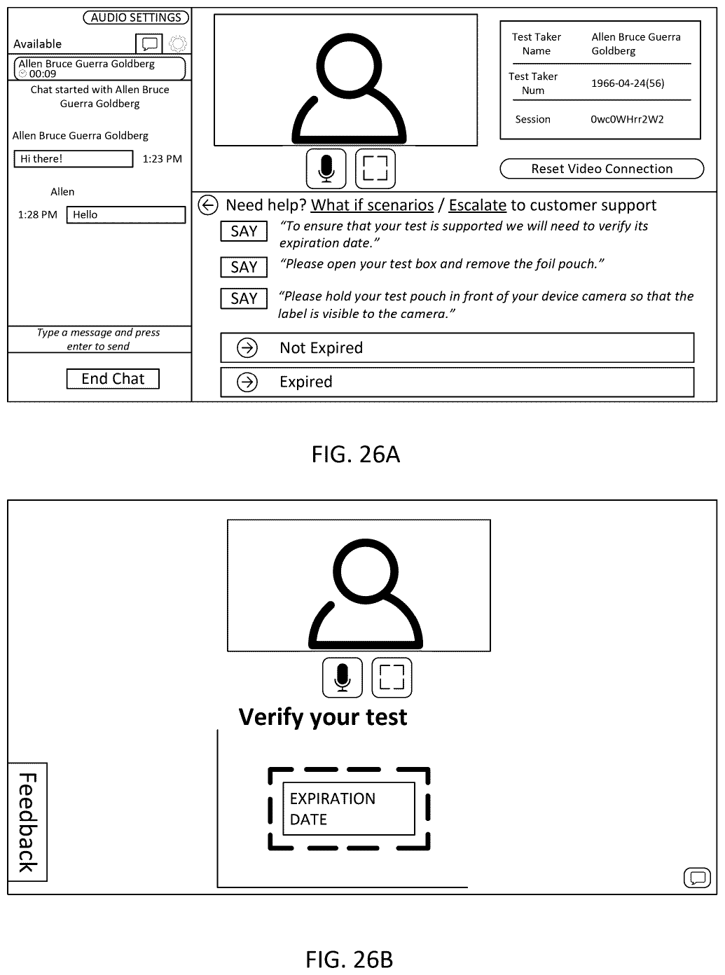

FIGS. 26A-26B show example side-by-side views of the proctor device display and the user device display as a drug test kit expiration date is being verified. The proctor may be prompted to instruct the user to hold the drug test kit in front of the camera so that the proctor may verify that the drug test kit is unopened or untampered with. In some embodiments, the proctor may also ask the patient to make the expiration date visible to the camera in order to verify that the drug test kit is not yet expired.

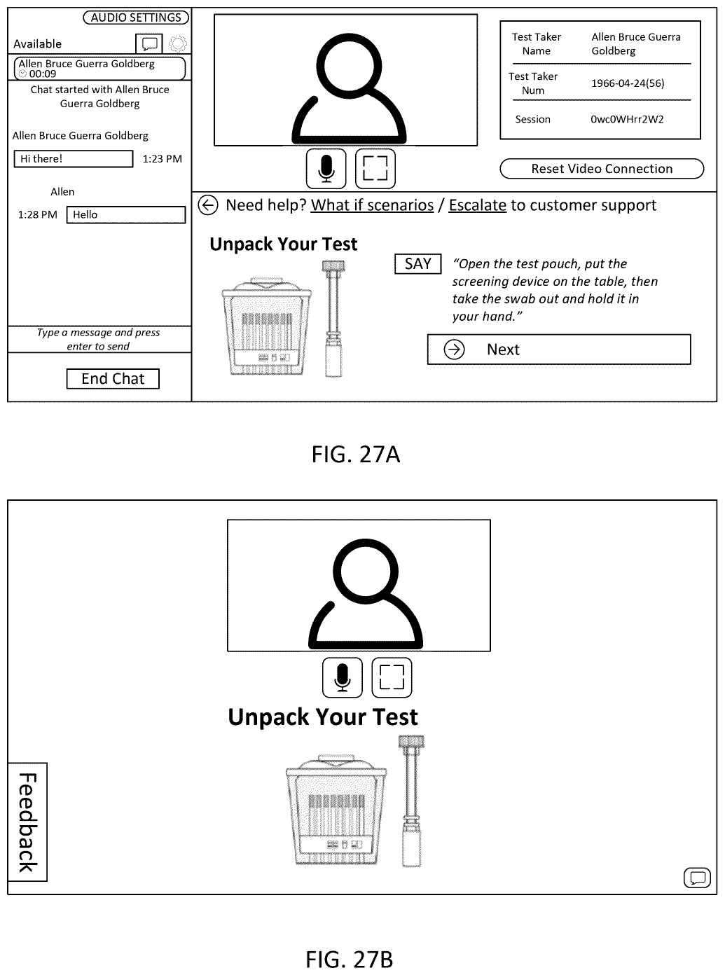

FIGS. 27A-27B shows example side-by-side views of the proctor device display and the user device display as the components of a drug test kit are being verified. The proctor may be prompted to ask the user to unpack and display all components of the drug test kit so as to confirm that all necessary components are present.

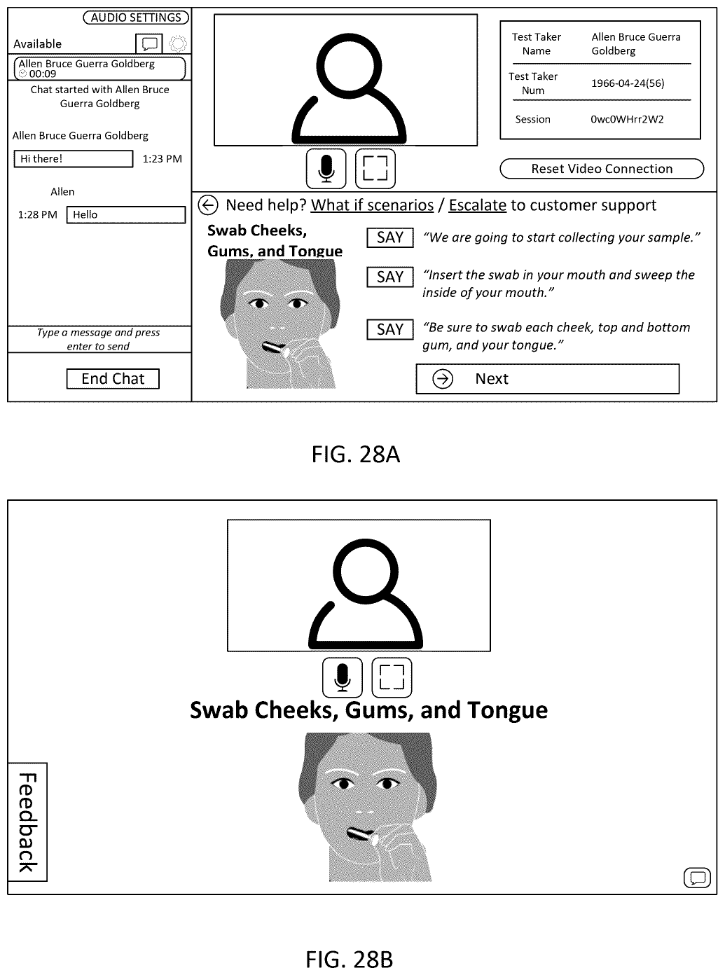

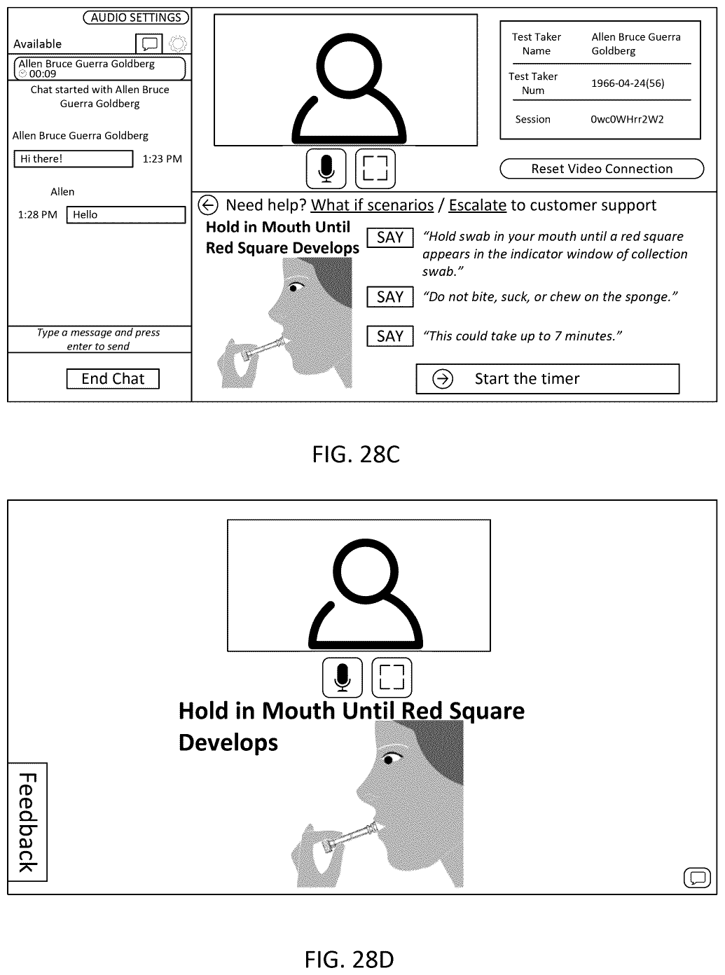

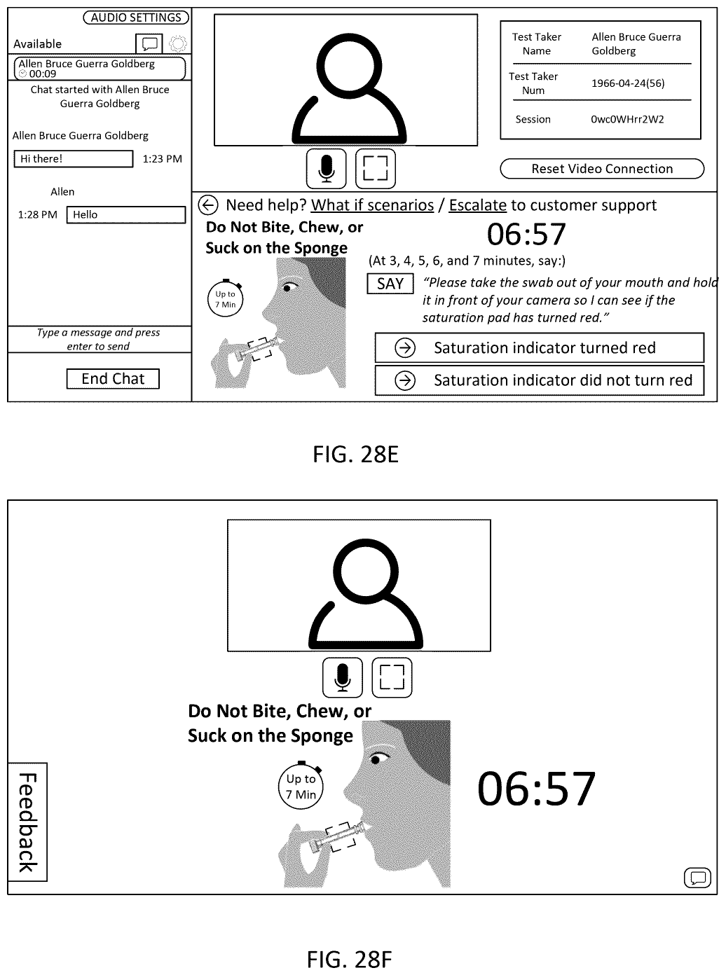

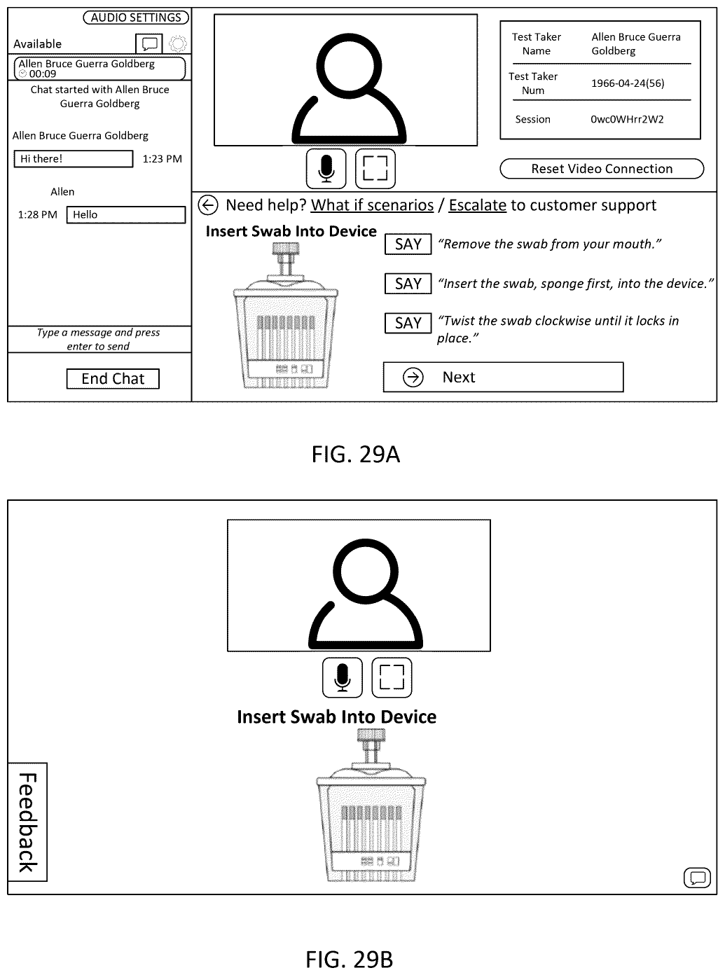

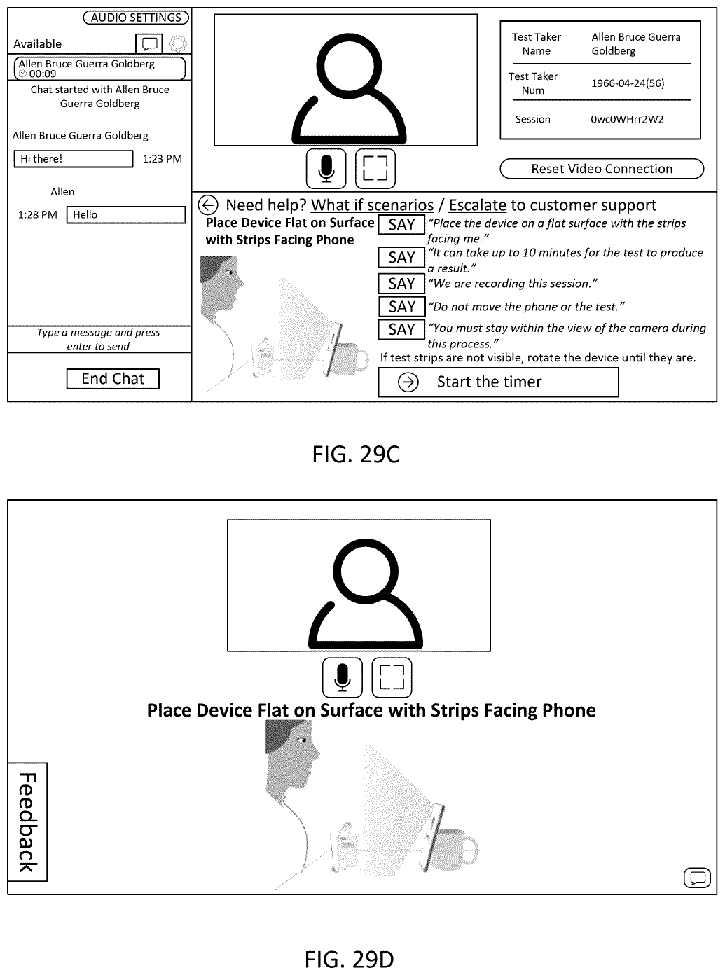

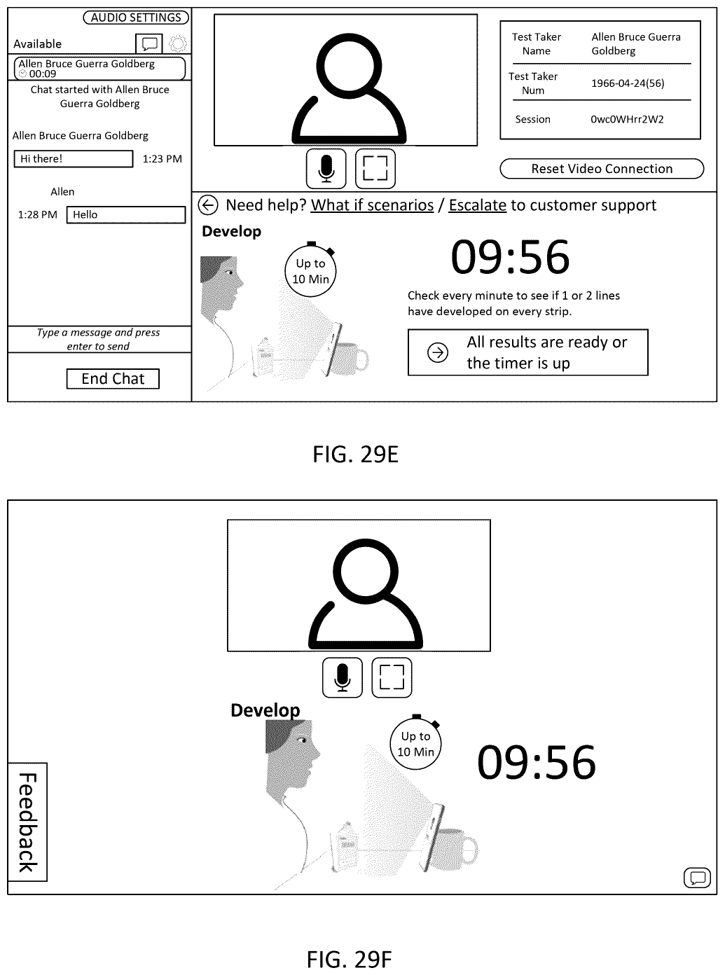

FIGS. 28A-28F show example side-by-side views of the proctor device display and the user device display during the sample collection stage of the testing experience. FIGS. 29A-29F show example side-by-side views of the proctor device display and the user device display during the sample testing stage of the testing experience. In some embodiments, the doctor may be able to see the graphic being shown on the user's display in addition to the scripted instructions.

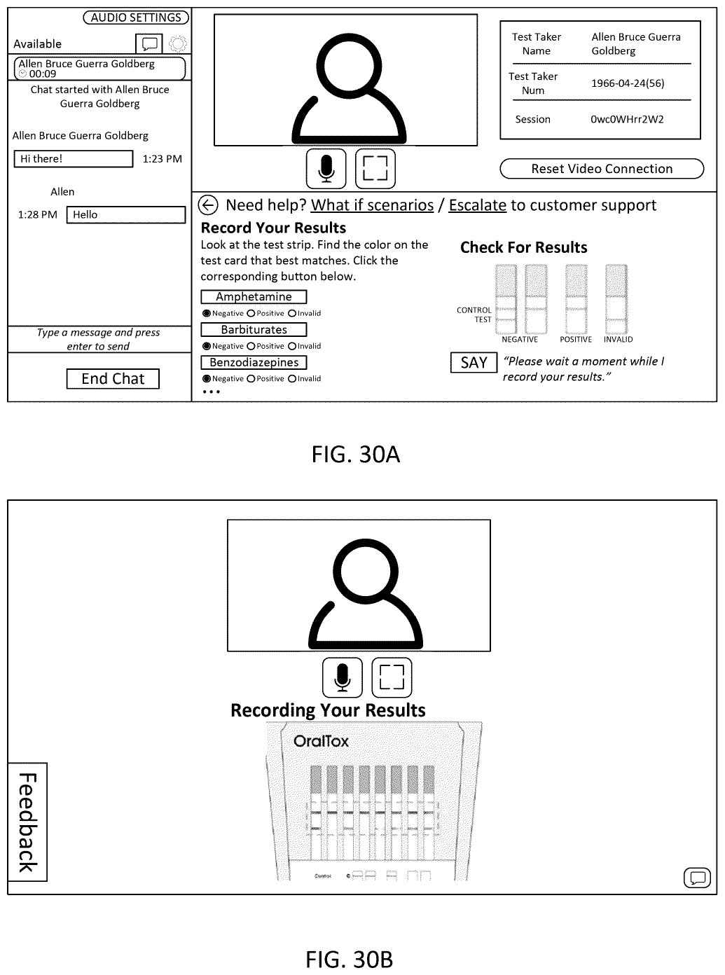

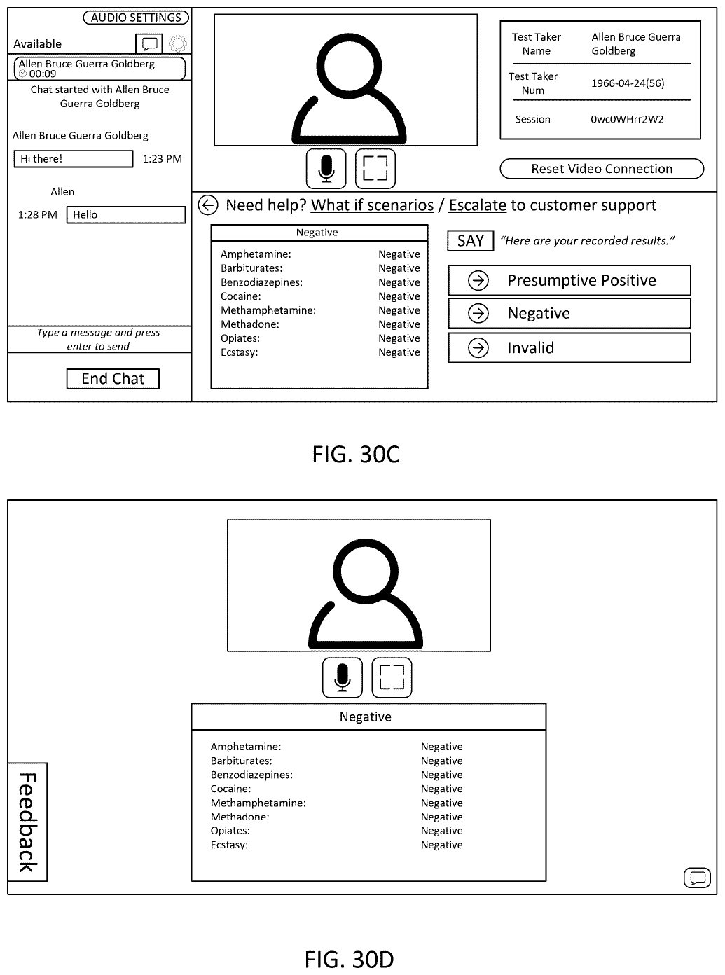

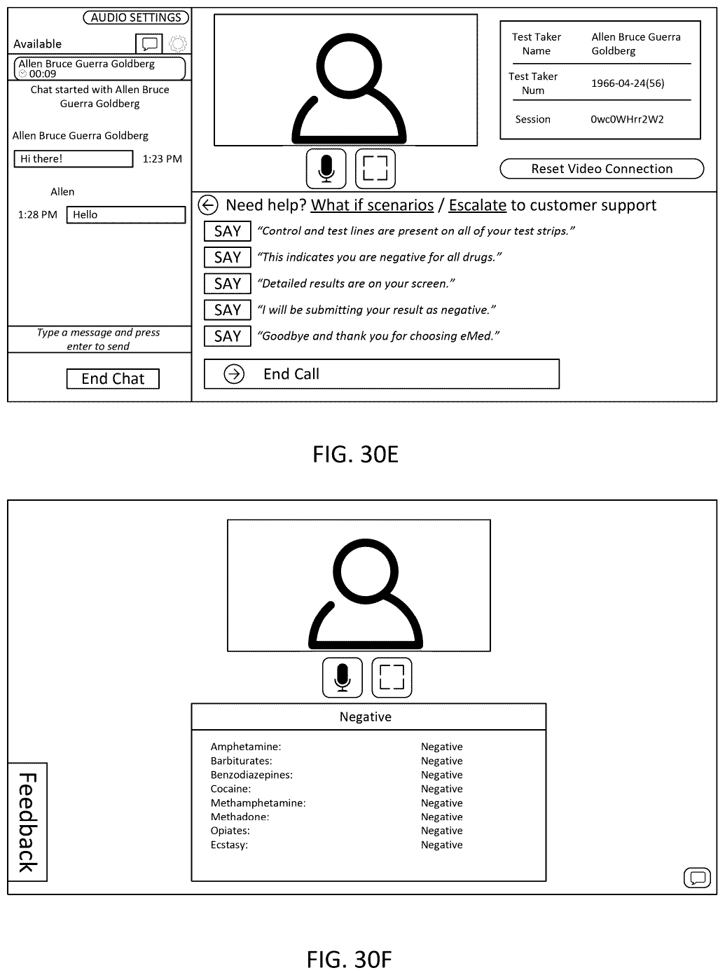

FIGS. 30A-30F show example side-by-side views of the proctor device display and the user device display during the results interpretation stage of the testing experience. In some embodiments, the proctor may be provided with a results interpretation guide along with scripted prompts to instruct the patient how to interpret the results. Having both the proctor and user interpret the results separately helps confirm the accuracy of the interpretation. For example, if both the proctor and the user come to the same interpretation, it is more likely that the interpretation is correct.

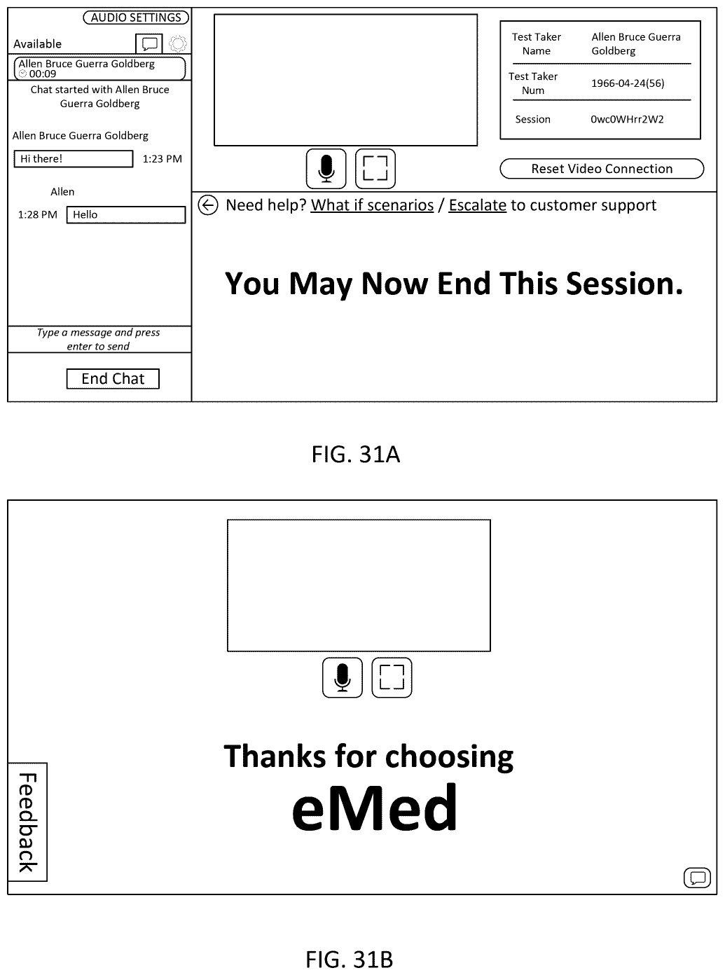

FIGS. 31A-31B show example side-by-side views of the proctor device display and the user device display at the conclusion of the testing experience.

### Computer Systems

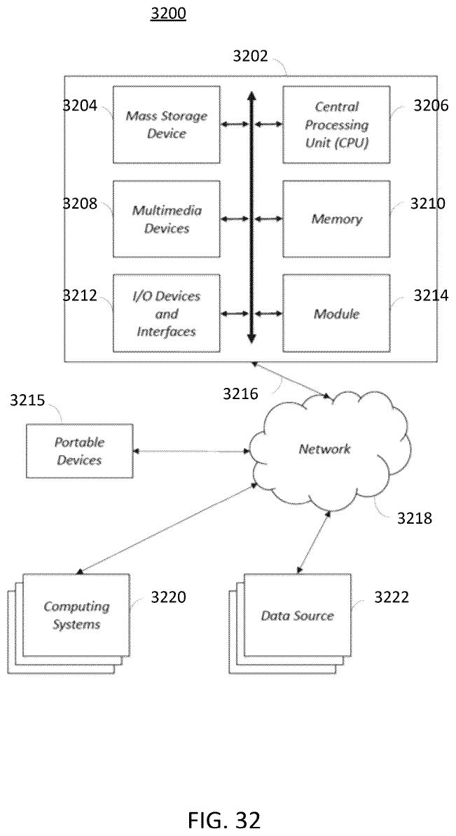

FIG. 32 is a block diagram depicting an embodiment of a computer hardware system configured to run software for implementing the systems, methods, and devices disclosed herein. The example computer system 3202 is in communication with one or more computing systems 3220 and/or one or more data sources 3222 via one or more networks 3218. While FIG. 32 illustrates an embodiment of a computing system 3202, it is recognized that the functionality provided for in the components and modules of computer system 3202 may be combined into fewer components and modules, or further separated into additional components and modules.

The computer system 3202 can comprise a module 3214 that carries out the functions, methods, acts, and/or processes described herein. The module 3214 is executed on the computer system 3202 by a central processing unit 3206 discussed further below.

In general, the word “module,” as used herein, refers to logic embodied in hardware or firmware or to a collection of software instructions, having entry and exit points. Modules are written in a program language, such as JAVA, C or C++, PYTHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 3202 includes one or more processing units (CPU) 3206, which may comprise a microprocessor. The computer system 3202 further includes a physical memory 3210, such as random-access memory (RAM) for temporary storage of information, a read only memory (ROM) for permanent storage of information, and a mass storage device 3204, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3202 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 3202 includes one or more input/output (I/O) devices and interfaces 3212, such as a keyboard, mouse, touch pad, and printer. The I/O devices and interfaces 3212 can include one or more display devices, such as a monitor, which allows the visual presentation of data to a user. More particularly, a display device provides for the presentation of GUIs as application software data, and multi-media presentations, for example. The I/O devices and interfaces 3212 can also provide a communications interface to various external devices. The computer system 3202 may comprise one or more multi-media devices 3208, such as speakers, video cards, graphics accelerators, and microphones, for example.

The computer system 3202 may run on a variety of computing devices, such as a server, a Windows server, a Structure Query Language server, a Unix Server, a personal computer, a laptop computer, and so forth. In other embodiments, the computer system 3202 may run on a cluster computer system, a mainframe computer system and/or other computing system suitable for controlling and/or communicating with large databases, performing high volume transaction processing, and generating reports from large databases. The computing system 3202 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 3202 illustrated in FIG. 32 is coupled to a network 3218, such as a LAN, WAN, or the Internet via a communication link 3216 (wired, wireless, or a combination thereof). Network 3218 communicates with various computing devices and/or other electronic devices. Network 3218 is communicating with one or more computing systems 3220 and one or more data sources 3222. The module 3214 may access or may be accessed by computing systems 3220 and/or data sources 3222 through a web-enabled user access point. Connections may be a direct physical connection, a virtual connection, and other connection type. The web-enabled user access point may comprise a browser module that uses text, graphics, audio, video, and other media to present data and to allow interaction with data via the network 3218.

Access to the module 3214 of the computer system 3202 by computing systems 3220 and/or by data sources 3222 may be through a web-enabled user access point such as the computing systems' 3220 or data source's 3222 personal computer, cellular phone, smartphone, laptop, tablet computer, e-reader device, audio player, or another device capable of connecting to the network 3218. Such a device may have a browser module that is implemented as a module that uses text, graphics, audio, video, and other media to present data and to allow interaction with data via the network 3218.

The output module may be implemented as a combination of an all-points addressable display such as a cathode ray tube (CRT), a liquid crystal display (LCD), a plasma display, or other types and/or combinations of displays. The output module may be implemented to communicate with input devices 3212 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 3202 may comprise a physical or logical connection established between a remote microprocessor and a mainframe host computer for the express purpose of uploading, downloading, or viewing interactive data and databases on-line in real time. The remote microprocessor may be operated by an entity operating the computer system 3202, including the client server systems or the main server system, an/or may be operated by one or more of the data sources 3222 and/or one or more of the computing systems 3220. In some embodiments, terminal emulation software may be used on the microprocessor for participating in the micro-mainframe link.

In some embodiments, computing systems 3220 who are internal to an entity operating the computer system 3202 may access the module 3214 internally as an application or process run by the CPU 3206.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 3202 may include one or more internal and/or external data sources (for example, data sources 3222). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 3202 may also access one or more databases 3222. The databases 3222 may be stored in a database or data repository. The computer system 3202 may access the one or more databases 3222 through a network 3218 or may directly access the database or data repository through I/O devices and interfaces 3212. The data repository storing the one or more databases 3222 may reside within the computer system 3202.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A computer-implemented method for remote drug testing, the method comprising:
receiving, from a user device, first user input data verifying that a user has not performed oral consumption for a period of time;
directing the user device to perform a check of components involved in the remote drug testing;
sending, to the user device, first user interface data for displaying instructions for the user to follow prior to beginning the remote drug testing;
receiving, from the user device, second user input data initiating the remote drug testing;
upon receiving the second user input data, establishing a video connection, an audio connection, and a real-time messaging service between the user device and a supervising user device;
sending, to the user device, second user interface data for displaying instructions to provide an image of a user identification via their user device;
facilitating transmittal, from the user device to the supervising user device, the image of the user identification;
receiving, from the supervising user device, a verification of the user identification;
sending, to the user device, an image of a test;
receiving, from the user device, third user input data confirming that the test matches a user test;
facilitating transmittal, from the user device to the supervising user device, an image of the user test;
receiving, from the supervising user device, a confirmation that the user test is not expired;
sending, to the user device, third user interface data for displaying instructions to collect a sample and instructions to develop results of the user test from the sample;
facilitating transmittal, from the user device to the supervising user device, results of the user test;
receiving, from the supervising user device, a recordation of the results of the user test;
generating a report based on the recordation of the results of the user test; and
sending the report to the user device.

2. The computer-implemented method of claim 1, wherein the supervising user device is a proctor device.

3. The computer-implemented method of claim 1, wherein the user identification is an identification card for the user.

4. The computer-implemented method of claim 1, wherein the method further comprises:
sending, to the user device, instructions illustrating components of the user test and explaining how the components will be used.

5. The computer-implemented method of claim 1, wherein the method further comprises:
sending the report to an employer, government agency, or an insurance agency.

6. The computer-implemented method of claim 1, wherein the method further comprises:
collecting video data from the user device during the remote drug testing; and
analyzing the video data to check that the user test is within view of a camera of the user device throughout the remote drug testing.

7. The computer-implemented method of claim 1, wherein the method further comprises:
collecting video data from the user device during the remote drug testing;
analyzing the video data to check that the user test is within view of a camera of the user device throughout the remote drug testing; and
upon determining that the user test is not within view of the camera of the user device throughout the remote drug testing, invalidating the results of the user test.

8. The computer-implemented method of claim 1, wherein the method further comprises:
sending, to the supervising user device, fourth user interface data for verifying the user identification.

9. The computer-implemented method of claim 1, wherein the method further comprises:
sending, to the supervising user device, fourth user interface data for confirming that the user test is not expired.

10. The computer-implemented method of claim 1, wherein the method further comprises:
sending, to the supervising user device, fourth user interface data for recording the results of the user test.

11. A non-transient computer readable medium containing program instructions for causing a computer to perform a method for remote drug testing, the method comprising:
receiving, from a user device, first user input data verifying that a user has not performed oral consumption for a period of time;
directing the user device to perform a check of components involved in the remote drug testing;
sending, to the user device, first user interface data for displaying instructions for the user to follow prior to beginning the remote drug testing;
receiving, from the user device, second user input data initiating the remote drug testing;
upon receiving the second user input data, establishing a video connection, an audio connection, and a real-time messaging service between the user device and a supervising user device;
sending, to the user device, second user interface data for displaying instructions to provide an image of a user identification via their user device;
facilitating transmittal, from the user device to the supervising user device, the image of the user identification;
receiving, from the supervising user device, a verification of the user identification;
sending, to the user device, an image of a test;
receiving, from the user device, third user input data confirming that the test matches a user test;
facilitating transmittal, from the user device to the supervising user device, an image of the user test;
receiving, from the supervising user device, a confirmation that the user test is not expired;
sending, to the user device, third user interface data for displaying instructions to collect a sample and instructions to develop results of the user test from the sample;
facilitating transmittal, from the user device to the supervising user device, results of the user test;
receiving, from the supervising user device, a recordation of the results of the user test;
generating a report based on the recordation of the results of the user test; and
sending the report to the user device.

12. The non-transient computer readable medium of claim 11, wherein the supervising user device is a proctor device.

13. The non-transient computer readable medium of claim 11, wherein the user identification is an identification card for the user.

14. The non-transient computer readable medium of claim 11, wherein the method further comprises:
sending, to the user device, instructions illustrating components of the user test and explaining how the components will be used.

15. The non-transient computer readable medium of claim 11, wherein the method further comprises:
sending the report to an employer, government agency, or an insurance agency.

16. The non-transient computer readable medium of claim 11, wherein the method further comprises:
collecting video data from the user device during the remote drug testing; and
analyzing the video data to check that the user test is within view of a camera of the user device throughout the remote drug testing.

17. The non-transient computer readable medium of claim 11, wherein the method further comprises:
collecting video data from the user device during the remote drug testing;
analyzing the video data to check that the user test is within view of a camera of the user device throughout the remote drug testing; and
upon determining that the user test is not within view of the camera of the user device throughout the remote drug testing, invalidating the results of the user test.

18. The non-transient computer readable medium of claim 11, wherein the method further comprises:
sending, to the supervising user device, fourth user interface data for verifying the user identification.

19. The non-transient computer readable medium of claim 11, wherein the method further comprises:
sending, to the supervising user device, fourth user interface data for confirming that the user test is not expired.

20. The non-transient computer readable medium of claim 11, wherein the method further comprises:
sending, to the supervising user device, fourth user interface data for recording the results of the user test.
